(19) United States
(12) Patent Application Publication
(10) Pub. No.: US 2012/0197214 A1
(43) Pub. Date: Aug. 2, 2012

(54) PERIPHERAL NERVE BLOCKADE DEVICE

(76) Inventors: Mark David Manley, Loveland, OH (US); Douglas Paul Stewart, Loveland, OH (US); Brian Newell Vaughan, Cincinnati, OH (US)

(21) Appl. No.: 13/019,762

(22) Filed: Feb. 2, 2011

(51) Int. Cl. A61M 19/00; A61M 5/00

(52) U.S. Cl. 604/246; 604/272

(57) ABSTRACT

A continuous nerve block apparatus comprising: (a) a hollow needle including a conduit extending between a proximal end and a distal end, the distal end being beveled between ten and fifty degrees; and, (b) a connector operatively coupled to the hollow needle, the connector including at least a first through passage in fluid communication with the conduit of the hollow needle, the connector including a first orifice allowing fluid communication from an exterior of the connector to the first through passage, the connector also including at least one valve.

### RELATED ART

1. Field of the Invention

The present disclosure is directed to methods and devices assisting with peripheral nerve blockade. The disclosure also relates to devices and methods for delivering fluid and local anesthetic medications to the tissues surrounding peripheral nerves.

2. Brief Discussion of Related Art

The technique of peripheral nerve blockade provides for extended pain relief to a region of a patient's body. Pain relief is accomplished by the delivery of local anesthetic into the tissue surrounding either sensory nerves, motor nerves, or both. When initially developed, peripheral nerve blocks were placed using only physical landmarks and patient's response (paresthesia). Since that time, the technique has been improved through the use of electrical nerve stimulation and ultrasound guidance for more accurate nerve localization as well as the placement of catheters for prolonged duration of action. Although the technique of peripheral nerve blockade has evolved, many of the instruments currently in use were designed for alternate purposes. The instant disclosure is directed to devices and methods developed specifically for peripheral nerve blocks to improve performance.

### INTRODUCTION TO THE INVENTION

The present invention is directed to methods and devices assisting with peripheral nerve blockade, as well as devices and methods for delivering fluid and local anesthetic medications to the tissues surrounding peripheral nerves.

It is a first aspect of the present invention to provide a continuous nerve block apparatus comprising: (a) a hollow needle including a conduit extending between a proximal end and a distal end, the distal end being beveled between ten and fifty degrees, and (b) a connector operatively coupled to the hollow needle, the connector including at least a first through passage in fluid communication with the conduit of the hollow needle, the connector including a first orifice allowing fluid communication from an exterior of the connector to the first through passage, the connector also including at least one valve.

In a more detailed embodiment of the first aspect, the hollow needle includes an ultrasound reflective coating. In yet another more detailed embodiment, the hollow needle includes a segment that is not smooth in order to increase reflection of ultrasound. In a further detailed embodiment, the hollow needle includes exterior incremental distance markings. In still a further detailed embodiment, the valve comprises a flexible diaphragm. In a more detailed embodiment, the embodiment further includes a flexible tube having a distal end and a proximal end, the proximal end coupled to a luer taper connection, and the distal end extending through the first orifice and into communication with the first through passage. In a more detailed embodiment, the embodiment further includes a second orifice extending from the through passage to the exterior of the connector. In another more detailed embodiment, the embodiment further includes a conductive stimulator line extending through the second orifice and into communication with the first through passage. In yet another more detailed embodiment, the connector includes a T-shaped portion having a pair of wings laterally extending from a handle. In still another more detailed embodiment, the embodiment further includes a catheter sized to extend through the first through passage and the hollow needle.

In yet another more detailed embodiment of the first aspect, the catheter is in fluid communication with a flexible fluid conduit upstream from the catheter. In still another more detailed embodiment, the catheter includes a conductive tip. In a further detailed embodiment, the catheter houses a wire in electrical communication with the conductive tip. In still a further detailed embodiment, the embodiment further includes a removable adapter receiving an end of the catheter, the wire, and a flexible fluid conduit upstream from the catheter. In a more detailed embodiment, the catheter extends through the at least one valve. In a more detailed embodiment, at least a portion of the catheter extends from a distal end of the needle and another portion of the catheter extends from a proximal end of the connector opposite the needle.

It is a second aspect of the present invention to provide a continuous nerve block apparatus comprising: (a) a hollow needle including a conduit extending between a proximal end and a distal end, the distal end being beveled between ten and fifty degrees; (b) a connector operatively coupled to the hollow needle, the connector including a first through passage that receives the hollow needle, the connector including a first orifice allowing fluid communication from an exterior of the connector and into the first through passage; and, a catheter comprising a hollow tube and a conductive distal end, the catheter having a length long enough to concurrently occupy the conduit of the hollow needle and the first through passage of the connector so the conductive distal end extends from the distal end of the needle and a portion of the catheter extends from a proximal end of the connector.

In a more detailed embodiment of the second aspect, the hollow needle includes an exterior surface that is specially modified to increase ultrasound reflectivity. In yet another more detailed embodiment, the hollow needle includes distance indicia on an exterior thereof and, the catheter includes distance indicia on an exterior thereof. In a further detailed embodiment, the hollow needle is in fluid communication with a first flexible conduit including a luer lock and, the catheter is in fluid communication with a second flexible conduit including a luer lock.

### BRIEF DESCRIPTION OF THE DRAWINGS

FIG. 1 is a top view of an exemplary component part of the peripheral nerve blockade delivery device.

FIG. 2 is a cross-sectional view of the exemplary component part of FIG. 1.

FIG. 3 is a top view of another exemplary component part of the peripheral nerve blockade delivery device.

### DETAILED DESCRIPTION

The exemplary embodiments of the present invention are described and illustrated below to encompass methods of printing images onto a substrate as well as goods having images printed thereon. Of course, it will be apparent to those of ordinary skill in the art that the preferred embodiments discussed below are exemplary in nature and may be reconfigured without departing from the scope and spirit of the present invention. However, for clarity and precision, the exemplary embodiments as discussed below may include optional steps, methods, and features that one of ordinary skill should recognize as not being a requisite to fall within the scope of the present invention.

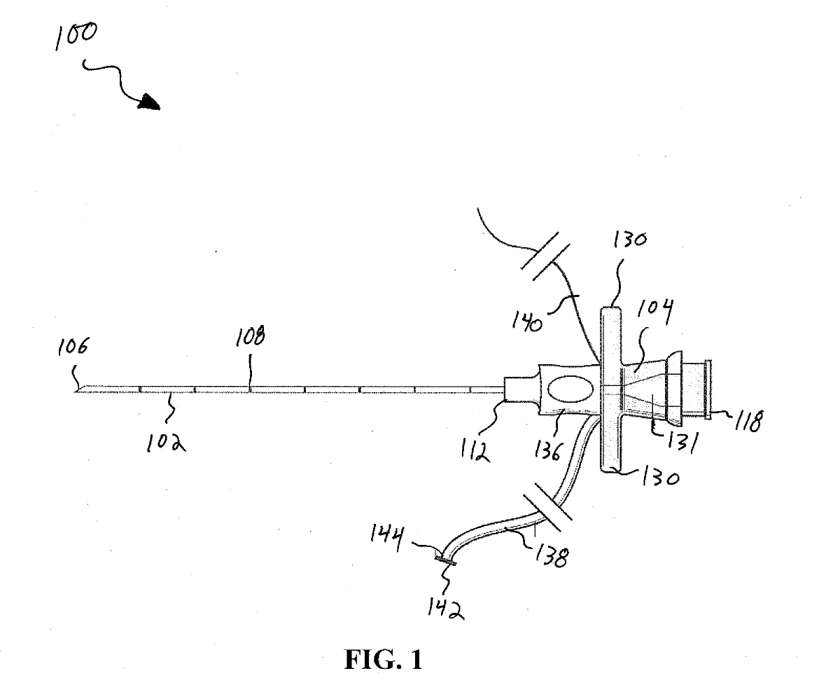

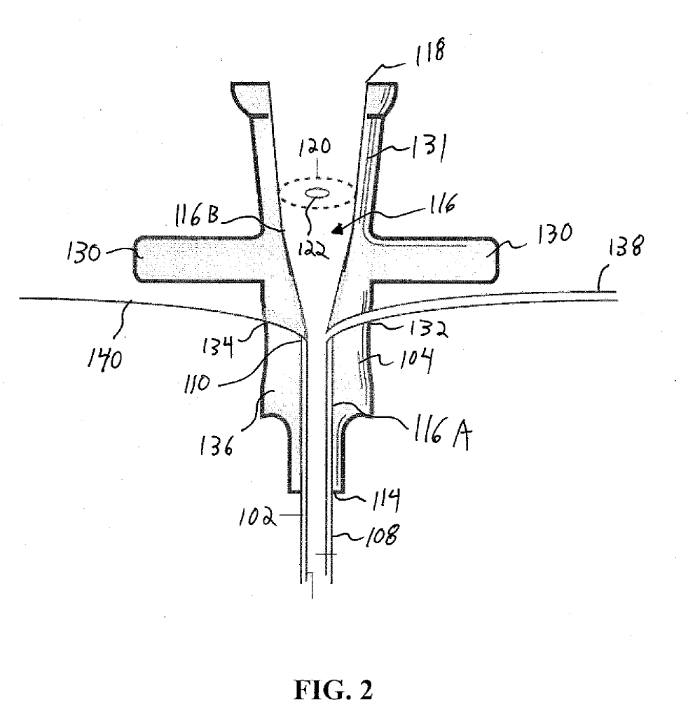

Referencing FIGS. 1-3, an exemplary peripheral nerve blockade delivery device 100 comprises a needle 102 coupled to a T-connector 104. The needle 102 comprises a hollow electrically conductive tube that may be fabricated from any biologically compatible metal or other biologically compatible material. A distal end 106 of the needle 102 includes a B-bevel point angled between ten and fifty degrees, for example. The duller point of the B-bevel, as opposed to a longer and sharper A-bevel, provides a practitioner (e.g., a anesthesiologist) with greater tactile sensation. At the same time the B-bevel allows for more controlled, precise tissue penetration as compared to the Tuohy needle that is currently used for the introduction of continuous catheters.

An exterior surface 108 of the needle 102 includes a coating that reflects ultrasound in order to view the needle during an ultrasound procedure. Alternatively, or in addition, the exterior surface 108 may be roughened by abrasion or the addition of surface projections operative to reflect a greater amount of ultrasound, thereby increasing the visibility of the needle 102 during an ultrasound procedure. Alternatively, or in addition, the exterior surface 108 may be coated with an insulating material to inhibit electrical communication across the exterior of the needle 102. Though not required, the exterior surface 108 may include distance markings, such as, without limitation, markings of the English measurement or metric measurements. Exemplary distance markings may be inscribed on the exterior surface 108 of the needle 102 to provide information to the practitioner (e.g., an anesthesiologist) as to the exact depth of the needle within a patient's tissue. In this exemplary embodiment, the needle has a length ranging between fifty to one hundred twenty millimeters. But is should also be understood that these lengths are exemplary in nature and other lengths longer than one hundred twenty millimeters and shorter than fifty millimeters may be utilized without departing from the scope and spirit of the instant disclosure. Likewise, various needle 102 diameters may be used as part of the exemplary peripheral nerve blockade delivery device 100. For example, needle 102 gauges such as 17 G, 18 G, and 19 G may be used with the embodiments of the instant disclosure. Nevertheless, these exemplary catheter gauges are to be understood as non-limiting as catheters larger than 19 G and smaller than 17 G may be used as part of the exemplary peripheral nerve blockade delivery device 100.

The needle 102 may be insulated or uninsulated. For motor/sensory nerve blocks, the body of the needle 102 is insulated so that only the distal end 106 is capable of passing electrical current from the needle into the patient's body. As will be discussed hereafter, a stimulator connector 140 is used to carry electrical current from a stimulator cord to an interior, conductive surface of the needle 102. Conversely, for sensory only nerve blocks, the needle 102 is uninsulated and the stimulator connector 140 may be omitted.

Opposite the distal end 106 of the needle 102, a proximal end 110 is coupled to the T-connector 104. The T-connector 104 is fabricated from an electrically insulative material and includes a distal end 112 having an orifice 114 that is sized to receive the proximal end 110 of the needle 102 in order to create a fluidic seal therebetween. Extending proximally within the T-connector 104, away from the needle 102 and the orifice 114, is a through conduit 116 that extends generally linearly and axially centered all the way to a proximal end 118. In this exemplary embodiment, the through conduit 116 has a first cylindrical section 116A with a generally constant diameter and circular cross-section. This first cylindrical section 116A abuts a first conical section 116E having a generally circular cross-section, but the diameter of the circular cross-section increases along the length thereof until reaching a maximum diameter at the proximal end 118. As will be discussed in more detail hereafter, a flexible diaphragm 120 extends across the through conduit 116 proximate the proximal end 118. This flexible diaphragm 120 includes a central opening 122 adapted to receive a catheter in order to allow the smooth introduction of the catheter into the needle 102. When not pierced, the central opening 122 is closed so as to form a valve that limits fluid leakage thereacross.

An exterior of the T-connector 104 includes a pair of wings 130 that are offset 180 degrees from one other and cooperate with a handle 131 in order for a practitioner to control and manipulate the needle 102. In front of (i.e., distal) the wings 130, are a pair of orifices 132, 134 that provide communication between the exterior of the T-connector 104 and the through conduit 116, just proximal to the end of the proximal end of the needle 102. In exemplary form, the orifices 132, 134 are positioned on opposite sides of a distal housing 136 and are occupied by a flexible tube 138 (e.g., a plastic, flexible tube) and an insulated wire 140. Both the flexible tube 138 and the insulated wire 140 may be any desired length.

The orifices 132, 134, the flexible tube 138, and the wire 140 allow for simultaneous injection and nerve stimulation during a nerve block. In this exemplary embodiment, the flexible tube is generally between twenty to thirty centimeters, for example. The flexible tube 138 allows for injection of fluid via the needle as needed during block placement. Exemplary fluids include anesthetic and inert fluid (e.g., saline, 5% dextrose). In this exemplary embodiment, the first flexible tubing includes a universal luer lock 142 located at its distal end 144 in order to allow for attachment of tubing or a syringe. The insulated wire 140 electrically connects a stimulator (not shown) with the interior of the needle 102. Specifically, the insulated wire 140 extends through the orifice 134 and into the interior of the needle 102. A distal portion of the wire 140 extending into the interior of the needle 102 is not insulated and is coupled to the interior of the needle in order to secure the distal end. The wire 140 carries electrical current from the stimulator to the proximal end of the needle, where the electric current travels along the needle until reaching the uninsulated tip.

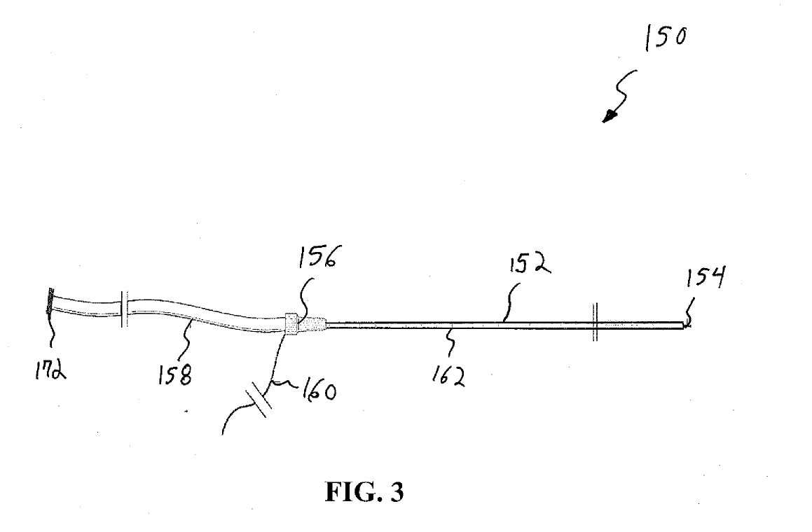

Referring specifically to FIG. 3, the exemplary peripheral nerve blockade delivery device 100 also comprises a continuous nerve block catheter assembly 150. The catheter assembly 150 includes a catheter 152, a conductive tip 154, a friction fit adapter 156, flexible tubing 158, and an insulated wire 160. The catheter 152 comprises a hollow, flexible and electrically conductive conduit with distance markings 162 in order to allow practitioners to precisely know the distance from each set distance marking along the catheter to the conductive tip 154. This catheter 152 may be fabricated from any biologically compatible material that is electrically conductive such as, without limitation silver, aluminum, copper, and stainless steel. In this exemplary embodiment, the catheter 152 is in fluid communication with the conductive tip 154, which includes at least one outlet orifice. It should be understood, however, that the conductive tip 154 may a single or multiple orifices, depending on the type of nerve block, to allow for proper distribution of fluid or local anesthetic.

The catheter 152 is insulated on its exterior and may be any length desired. In exemplary form, the insulated exterior may be comprised of a flexible non-conductive material such as, without limitation, plastic, rubber, and a polymer (e.g., polyurethane). A proximal end of the catheter 152 is received within a through conduit of the adapter 156 in order to couple the catheter to the adapter. In this exemplary embodiment, the adapter 156 comprises an elastomeric material that circumscribes the catheter 152 to form a seal therebetween and mount the adapter to the catheter. An opposite end of the adapter 156 is enlarged so that the diameter of the through conduit is substantially larger to accommodate insertion of the flexible tubing 158 and the insulated wire 160. Again, the elastic properties of the adapter 156 concurrently circumscribe the flexible tubing 158 and the insulated wire 160 to form a seal therebetween, while at the same time retaining the flexible tubing and the insulated wire within the adapter. The distal end of the flexible tubing 158 terminates within the through conduit, while the insulated wire extends through the adapter 156 and through the catheter 150 and into electrical communication with the conductive tip 154. A proximal end of the flexible tubing 158 includes a universal luer lock 172, while the proximal end of the insulated wire 160 is adapted to be coupled to a stimulator (not shown).

An exemplary procedure for using the peripheral nerve blockade delivery device 100 includes a practitioner grasping the T-connector 104 and orienting the distal end 106 of the needle 102 away from the practitioner. The distal end 106 of the needle 102 is inserted through a patient's tissue to a predetermined depth using ultrasound in combination with the distance markings on the exterior 108 of the needle. After the distal end 106 of the needle 102 is positioned, anesthetic and any inert fluid is injected through the distal end 106 of the needle by supplying such fluid through the flexible tube 138 and into communication with the interior of the needle by way of the proximal end. Because the distal end 106 of the needle 102 is uninsulated, electrical current may flow through the distal end by way of electrical communication between the needle and a stimulator. As discussed above, the wire 140 provides electrical communication between the stimulator and the interior of the needle, which is itself conductive. In this manner, when the stimulator is operative to disseminate and electric current into the wire 140, the result is electric current flowing through the distal end 140. This electric current flowing through the distal end may be used for nerve or muscle stimulation.

After the needle 102 has been positioned, the catheter 152 may be inserted through the proximal orifice 114 of the T-connector 104 and into the through conduit 116. The catheter 152 is inserted, conductive tip 154 first, and extends beyond the through conduit 116 and into the interior of the needle 102. In this exemplary embodiment, the catheter 152 is longer than the combined length of the T-connector 104 and the needle 102. This means that when the conductive tip 154 reaches the distal end of the needle 102, a portion of the catheter extends proximally outward from the proximal end of the T-connector. After the catheter 152 has been positioned so that the conductive tip 154 extends beyond the distal end 106 of the needle 102, anesthetic and any inert fluid is injected through the conductive tip 154 by supplying such fluid through the flexible tube 158 and into communication with the interior of the catheter. Because the conductive tip 154 of the catheter 152 is uninsulated, electrical current may flow through the conductive tip by way of electrical communication between the conductive tip and a stimulator. As discussed above, the insulated wire 160 is in electrical communication with the stimulator and the wire extends along the interior of the catheter 152 until reaching the conductive tip 154. In this manner, when the stimulator is operative to disseminate and electric current into the wire 160, the result is electric current flowing through the conductive tip 154. This electric current flowing through the conductive tip 154 may be used for nerve or muscle stimulation.

Following from the above description and invention summaries, it should be apparent to those of ordinary skill in the art that, while the methods and apparatuses herein described constitute exemplary embodiments of the present invention, the invention contained herein is not limited to this precise embodiment and that changes may be made to such embodiments without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

What is claimed is:

1. A continuous nerve block apparatus comprising:
a hollow needle including a conduit extending between a proximal end and a distal end, the distal end being beveled between ten and fifty degrees; and,
a connector operatively coupled to the hollow needle, the connector including at least a first through passage in fluid communication with the conduit of the hollow needle, the connector including a first orifice allowing fluid communication from an exterior of the connector to the first through passage, the connector also including at least one valve.

2. The continuous nerve block apparatus of claim 1, wherein the hollow needle includes an ultrasound reflective coating.

3. The continuous nerve block apparatus of claim 1, wherein the hollow needle includes a segment that is not smooth in order to increase reflection of ultrasound.

4. The continuous nerve block apparatus of claim 1, wherein the hollow needle includes exterior incremental distance markings.

5. The continuous nerve block apparatus of claim 1, wherein the valve comprises a flexible diaphragm.

6. The continuous nerve block apparatus of claim 1, further comprising a flexible tube having a distal end and a proximal end, the proximal end coupled to a luer taper connection, and the distal end extending through the first orifice and into communication with the first through passage.

7. The continuous nerve block apparatus of claim 1, further comprising a second orifice extending from the through passage to the exterior of the connector.

8. The continuous nerve block apparatus of claim 7, further comprising a conductive stimulator line extending through the second orifice and into communication with the first through passage.

9. The continuous nerve block apparatus of claim 1, wherein the connector includes a T-shaped portion having a pair of wings laterally extending from a handle.

10. The continuous nerve block apparatus of claim 1, further comprising a catheter sized to extend through the first through passage and the hollow needle.

11. The continuous nerve block apparatus of claim 10, wherein the catheter is in fluid communication with a flexible fluid conduit upstream from the catheter.

12. The continuous nerve block apparatus of claim 10, wherein the catheter includes a conductive tip.

13. The continuous nerve block apparatus of claim 12, wherein the catheter houses a wire in electrical communication with the conductive tip.

14. The continuous nerve block apparatus of claim 13, further comprising a removable adapter receiving an end of the catheter, the wire, and a flexible fluid conduit upstream from the catheter.

15. The continuous nerve block apparatus of claim 1, wherein the catheter extends through the at least one valve.

16. The continuous nerve block apparatus of claim 15, wherein at least a portion of the catheter extends from a distal end of the needle and another portion of the catheter extends from a proximal end of the connector opposite the needle.

17. A continuous nerve block apparatus comprising:
a hollow needle including a conduit extending between a proximal end and a distal end, the distal end being beveled between ten and fifty degrees;
a connector operatively coupled to the hollow needle, the connector including a first through passage that receives the hollow needle, the connector including a first orifice allowing fluid communication from an exterior of the connector and into the first through passage; and,
a catheter comprising a hollow tube and a conductive distal end, the catheter having a length long enough to concurrently occupy the conduit of the hollow needle and the first through passage of the connector so the conductive distal end extends from the distal end of the needle and a portion of the catheter extends from a proximal end of the connector.

18. The continuous nerve block apparatus of claim 17, wherein the hollow needle includes an exterior surface that is specially modified to increase ultrasound reflectivity.

19. The continuous nerve block apparatus of claim 17, wherein:
the hollow needle includes distance indicia on an exterior thereof; and,
the catheter includes distance indicia on an exterior thereof.

20. The continuous nerve block of claim 17, wherein:
the hollow needle is in fluid communication with a first flexible conduit including a luer lock; and,
the catheter is in fluid communication with a second flexible conduit including a luer lock.
